(19) United States
(12) Patent Application Publication
(10) Pub. No.: US 2020/0297593 A1
(43) Pub. Date: Sep. 24, 2020

(54) HYDROGEN PEROXIDE FORMULATIONS IN BARRIER LAYER FILMS WITH A SIOx LAYER

(71) Applicant: Henkel AG & Co. KGaA, Duesseldorf (DE)

(72) Inventors: MARC NOWOTTNY, Moenchengladbach (DE); TORSTEN LECHNER, Langenfeld (DE); WOLFGANG BARTHEL, Langenfeld (DE)

(73) Assignee: Henkel AG & Co. KGaA, Duesseldorf (DE)

(21) Appl. No.: 16/768,502

(22) PCT Filed: Nov. 12, 2018
(86) PCT No.: PCT/EP2018/080864

§ 371 Date: May 29, 2020

(30) Foreign Application Priority Data
Dec. 18, 2017 (DE) 10 2017 223 056.8

(51) Int. Cl. A61K 8/19; A61K 8/34; A61Q 5/10; A61K 8/49; A61K 8/55; A61K 8/368; A61K 8/92; B32B 27/08; B32B 27/36; B32B 27/32; A45D 34/00

(52) U.S. Cl. CPC A61K 8/19; B32B 2255/20; A61Q 5/10; A61K 8/4926; A61K 8/345; A61K 8/55; A61K 8/368; A61K 8/92; B32B 27/08; B32B 27/36; B32B 27/32; A45D 34/00; B32B 2553/00; B32B 2250/24; B32B 2255/10; A61K 8/342

(57) ABSTRACT

The present disclosure concerns a cosmetic product for modifying the natural color of keratinous fibers, in particular human hair, comprising at least one packaging (VP) and a cosmetic composition (KM) contained in this packaging (VP). The packaging is made of a multi-layer film (F) comprising at least two polymer layers (P1) and (P2) and at least one barrier layer (BS). The cosmetic composition comprises at least one oxidizing agent, at least one C₈-C₃₀ alcohol and at least one non-ionic surfactant. The use of the packaging (VP) in combination with the cosmetic composition (KM) does surprisingly not lead to an inflation of the packaging or an excessive loss of water of the agent (KM) during storage.

### CROSS-REFERENCE TO RELATED APPLICATION

This application is a U.S. National-Stage entry under 35 U.S.C. § 371 based on International Application No. PCT/EP2018/080864, filed Nov. 12, 2018, which was published under PCT Article 21(2) and which claims priority to German Application No. 10 2017 223 056.8, filed Dec. 18, 2017, which are all hereby incorporated in their entirety by reference.

### TECHNICAL FIELD

The present disclosure is in the field of cosmetics and relates to a product for oxidative color change of keratinous fibers, in particular human hair, which comprises a composition containing an oxidizing agent packed in a package. The oxidant-containing composition contains at least one C₈-C₃₀ alcohol and at least one non-ionic surfactant. The packaging is a package manufactured from a special multi-layer film composite system, the wall of which comprises at least two polymeric layers and a barrier layer. The barrier layer has a permeation barrier effect for gases and water vapor. The barrier layer comprises a silicon oxide.

### BACKGROUND

Changing the color of keratinous fibers, especially of hair, is an important area of modern cosmetics. The appearance of hair may be adjusted to current fashion trends as well as to the individual wishes of each person. The expert knows different methods to change the color of hair. The hair color may be temporarily changed by the use of directly drawing dyes. In this process, already formed colorants diffuse from the dye into the hair fiber. Dyeing with direct drawing dyes is associated with little damage to the hair, but a disadvantage is the low durability and the fast washability of the colorants obtained with direct drawing dyes.

If the consumer wants a long-lasting color result or a shade that is lighter than the original hair color, oxidative color changing products are usually used. For permanent, intensive dyeings with corresponding fastness properties, so-called oxidative coloring products are used. Such dyes usually contain oxidation dye precursors, so-called developer components and coupler components, which form the actual dyes under the effect of oxidizing agents—usually hydrogen peroxide. Oxidation dyes are exemplified by excellent, long-lasting dyeing results.

Oxidative color changing agents are usually marketed in the form of two-component products, in which two different preparations are separately packaged in two separate packages and are not mixed together until shortly before use. The first preparation is a formulation—usually acidic for stability reasons—which contains, for example, hydrogen peroxide as an oxidizing agent in concentrations of 1.5 to 12% by weight. The oxidizing agent formulation is usually in the form of an emulsion or dispersion and is usually provided in a plastic bottle with a resealable outlet opening (developer bottle).

This oxidizing agent formulation is mixed with a second preparation before use. This second preparation is an alkaline formulation which is often in the form of a cream or a gel and which, if a color change is desired at the same time as brightening, also contains at least one oxidation dye precursor. This second formulation may be provided, for example, in the form of a tube or in the form of a plastic or glass container.

In the usual method of application described above, the second preparation containing the alkalizing agent and/or the oxidation dye precursors is transferred from the tube or container into the developer bottle and then mixed by shaking with the hydrogen peroxide preparation already in the developer bottle. In this way, the application mixture is prepared in the developer bottle. The application to the hair is then effected via a small spout or outlet opening at the head of the developer bottle. The spout or outlet opening is opened after shaking and the application mixture is removed by pressing the flexible developer bottle.

The use of the developer bottle requires a certain routine from the user, so that some users prefer to prepare the application mixture in a mixing bowl and apply it with a brush.

When preparing the application mixture in a mixing bowl, both components—the first preparation containing the oxidizing agent and the second preparation containing an alkalizing agent and/or oxidation dye precursors—are transferred completely into a bowl or similar vessel and mixed there, for example, with the aid of a brush. The application mixture is then removed from the mixing bowl using the brush. With this form of application, the use of a voluminous and expensive developer bottle is not necessary, and the search continues for inexpensive and material-saving packaging forms for the oxidizing agent preparation.

In this context, packaging in the form of bags or pouches, which are usually made of plastic films or metal foils, is an inexpensive form of packaging with low material consumption.

One type of packaging may be produced, for example, by gluing or hot pressing two plastic foils one on top of the other, with the gluing taking place on all edges of the foils. The interior of the packaging (i.e. the plastic bag) created by the bonding may then be filled with the desired cosmetic preparation. The packaging may be opened by tearing or cutting open the plastic bag.

However, filling oxidizing agent preparations into such packaging is associated with problems caused by the reactivity of the oxidizing agent. Oxidizing agents are highly reactive substances which, depending on the storage conditions and the presence of decomposing impurities, decompose in small amounts to form oxygen (i.e. gas).

The developer bottles known from the state of the art are usually only filled with the oxidizing agent composition to a maximum of half, usually only to a third of their internal volume. As a rule, developer bottles are made of polyethylene. Since polyethylene is permeable to both water vapor and other gases, there is little or no excess pressure in the developer bottle. In addition, developer bottles are usually equipped with stable, thick walls and a sturdy screw cap, so that the diffusion of water vapor or gases through the thickness of the walls is reduced and a slight increase in pressure within the bottle has no negative effects.

In contrast, bag-shaped packaging is usually completely filled with the liquid preparation, and there is practically no excess air space in the filled bag. In addition, such packaging should be flexible, and when opened (e.g. torn open or cut open) there should be no uncontrolled escape of the preparation. For this reason, when packaging liquid preparations, the creation of excess pressure in the packaging should be avoided, if possible.

If an oxidizing agent preparation is in such a package, the gas (oxygen) produced during storage may cause the package to expand. Since the edges of the packaging are usually only glued together, in the worst case, a strong expansion can lead to bursting of the packaging. For these reasons, the choice of the film material of which the packaging is made is of great importance when storing oxidizing agent-containing preparations.

Packaging made of pure plastic such as polyethylene or polypropylene is permeable to both water vapor and gases. When storing oxidizing agent-containing preparations in polyethylene or polypropylene packaging, the packaging does not expand. Due to the high permeability of the comparatively thin film of the packaging to water vapor, however, the water content of the preparation is reduced. If the preparation is stored in the packaging for several weeks or months, the water loss exceeds the maximum value permitted for adequate storage stability.

The manufacture of suitable packaging for hydrogen peroxide-containing formulations is a challenge. The above properties for the permeability of oxygen and water vapor must be adjusted to ensure adequate storage stability. The thickness of the film layer should be kept as low as possible for environmental reasons and to spare resources. Furthermore, the layer thickness naturally also has an influence on the manufacturing costs. Against this background, thin layers are desired, but these do not always guarantee sufficient mechanical strength. If different materials are combined in a multi-layer film in order to satisfy a wide range of requirements, the manufacturability of the multi-layer film must also be guaranteed. Certain materials cannot be combined with each other because the cohesion between layers is not always sufficient or because their processing temperatures may be so different that joint processing is difficult.

Finally, the film materials are of great importance, especially when storing a multi-component system, as substances from the multi-component system may diffuse into the films and promote the detachment of layers forming the film. The choice of components in a hydrogen peroxide containing formulation therefore also has an influence on the choice of packaging.

### BRIEF SUMMARY

Cosmetic products for modifying the natural color of keratinous fibers are provided herein. In an embodiment, a cosmetic product includes (i) at least one packaging (VP) and (ii) at least one cosmetic composition (KM) packaged in the packaging (VP). The at least one packaging (i) includes at least one multi-layer film (F). The multi-layer film (F) includes at least one first polymer layer (P1), at least one second polymer layer (P2) and at least one barrier layer (BS). The at least one cosmetic composition (KM) includes a) at least one oxidizing compound, b) at least one C8-C30 alcohol, and c) at least one non-ionic surfactant. The first polymer layer (P1) is formed from polyethylene terephthalate or polyethylene naphthalate, the second polymer layer (P2) is formed from a polyolefin, and the barrier layer (BS) is formed from a polyester provided with an SiOₓ layer.

In another embodiment, a cosmetic product for modifying the natural color of keratinous fibers includes (i) at least one packaging (VP) and (ii) at least one cosmetic composition (KM) packaged in the packaging (VP). The at least one packaging (i) includes at least one multi-layer film (F). The multi-layer film (F) includes at least one first polymer layer (P1), at least one second polymer layer (P2) and at least one barrier layer (BS). The first polymer layer (P1) is formed from polyethylene terephthalate, the second polymer layer (P2) is formed from polyethylene, and the barrier layer (BS) is formed from a polyethylene terephthalate provided with an SiOₓ layer. The at least one cosmetic composition (KM) includes a) at least one oxidizing compound in a total amount of from about 1.5 to 15% by weight, relative to the total weight of the cosmetic composition (KM). The at least one cosmetic composition (KM) further includes b) a mixture of linear C₁₆-C₁₈ alcohols in a total amount of from about 1.5 to about 5.0% by weight, based on the total weight of the cosmetic composition (KM). The at least one cosmetic composition (KM) further includes c) a mixture of linear C₁₆-C₁₈ alcohols substituted with an average of 20 ethoxy groups in a total amount of from about 0.50 to 1.5% by weight, based on the total weight of the cosmetic composition (KM).

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The task of the present application was to package hydrogen peroxide-containing formulations in such a way that the mechanical strength of the packaging is sufficiently high to allow safe storage, but that easy access to the ingredients is ensured.

Surprisingly, it has now become apparent that oxidizing agent-containing formulations may be packaged in which the water vapor permeability is low and swelling may be reduced by allowing the film to have a certain degree of oxygen permeability. The films include a special film composite system and also has a barrier layer. By reducing the water vapor permeability, but adjusting the oxygen permeability to a sufficiently high level, the tendency to expand due to oxygen formed from hydrogen peroxide is reduced and the mechanical strength is increased over time.

The subject-matter of the present disclosure is a cosmetic product for modifying the natural color of keratinous fibers, in particular human hair, comprising
(i) at least one package (VP) comprising at least one multi-layer film (F) comprising at least one first polymer layer (P1), at least one second polymer layer (P2) and at least one barrier layer (BS), and
(ii) at least one cosmetic composition (KM) packaged and contained in the packaging (VP):
   a) at least one oxidizing compound,
   b) at least one C₈-C₃₀ alcohol, and
   c) at least one non-ionic surfactant,
   wherein the first polymer layer (P1) is composed of polyethylene terephthalate or polyethylene naphthalate, in particular of polyethylene terephthalate; the second polymer layer (P2) is composed of a polyolefin, in particular polyethylene; and the barrier layer (BS) is composed of a polyester provided with an SiOₓ layer, in particular of a polyethylene terephthalate provided with an SiOₓ layer.

Keratinous fibers, keratin-containing fibers or keratin fibers are understood to be furs, wool, feathers and in particular human hair. Although the products as contemplated herein are primarily suitable for lightening and dyeing keratinous fibers, there is nothing in principle to prevent their use in other areas.

The product as contemplated herein is a product for the oxidative color change of keratinous fibers, i.e. a product which is applied on human head to achieve an oxidative coloring, a lightening, a bleaching, or a nuance of the hair. In this context, nuance is understood to be a coloring in which the color result is lighter than the original hair color. That the product is to be used “to change the natural color” shall mean that the product either comprises only an oxidizing agent for bonding, or that the product comprises an oxidizing agent used with a conventional coupler to achieve a color change, or that the product is used with a conventional dye for further coloration.

Furthermore, as contemplated herein, the term “packaging” is understood to mean packaging which is preferably in the form of a sachet. A sachet is a small packaging in pocket or bag form, which is often used for the packaging of cosmetics. The capacity of the packaging, in particular of the sachet, may be, for example, from about 5 to about 1000 ml, preferably from about 10 to about 200 ml and particularly preferably from about 20 to about 50 ml.

In addition, a multi-layer film (F) in the context of the present disclosure is understood to be a thin, flat and roll-up sheet including the at least one polymer layer (P1) and the at least one polymer layer (P2). This multi-layer film (F) forms the wall of the package (VP). The packaging also comprises a barrier layer (BS), which selectively permits or reduces the passage of water vapor and other gases such as oxygen.

The cosmetic product as contemplated herein comprises as the first component a packaging (VP) which comprises at least one multi-layer film (F). This film comprises at least one first polymer layer (P1), at least one second polymer layer (P2) and at least one barrier layer (BS). This multi-layer film forms the wall or the outer shell of the packaging. As described above, this type of packaging is usually produced by gluing, pressing or welding two pieces of film on top of each other (the packaging (VP) is filled at the same time as the cosmetic composition (KM) is filled), i.e. such packaging is closed at all edges. This packaging may be opened, for example, by tearing or cutting it open.

The thickness of the multi-layer film (F) determines the mechanical properties and strength of the films. It should be designed in such a way that there is sufficient mechanical stability, but at the same time the film (F)—and thus the packaging (VP) made from the film—should be flexible enough to allow complete removal of the cosmetic composition (KM) from the opened packaging (VP) by pressing or squeezing. A film meets these requirements if the film (F) has a certain overall thickness. Preferred embodiments of the present disclosure are exemplified in that at least one multi-layer film shows a total thickness of from about 28 μm to about 220 μm, preferably of from about 52 μm to about 180 μm, more preferably of from about 80 μm to about 140 μm. For the purposes of the present disclosure, the total thickness of the film (F) is understood to be the sum of the thicknesses of all individual layers of the film (F).

The configuration of the layers (P1), (P2) and (BS) within the multi-layer film (F) may vary. Furthermore, it is also possible that the film (F) includes further layers in addition to the layers mentioned above. In addition, as contemplated herein, it is advantageous if all the previously mentioned layers are oriented parallel to the surfaces of the film (F), i.e. all layers have the same orientation. If the multi-layer film (F) contains the three layers (P1), (P2) and (BS) described above, the following configurations of the layers would be possible (viewed from the interior (in contact with the cosmetic composition (KM)) to the exterior):
a) *interior*-layer (P1)-layer (P2)-barrier layer (BS)-*exterior*,
b) *interior*-layer (P1)-barrier layer (BS)-layer (P2)-*exterior*,
c) *interior*-layer (P2)-layer (P1)-barrier layer (BS)-*exterior*,
d) *interior*-layer (P2)-barrier layer (BS)-layer (P1)-*exterior*,
e) *interior*-barrier layer (BS)-layer (P1)-layer (P2)-*exterior*,
f) *interior*-barrier layer (BS)-layer (P2)-layer (P1)-*exterior*.

However, as contemplated herein, it is preferred when the barrier layer (BS) is located between the first polymer layer (P1) and the second polymer layer (P2), the second polymer layer (P2) being located on the outside of the package. In this case, the multi-layer film (F) is composed of three layers, layer (P1) being at the innermost and in contact with the cosmetic composition (KM). Layer (P1) is in contact with the barrier layer (BS) and barrier layer (BS) is in contact with layer (P2). In this layer, layers (P1) and (P2) are not adjacent to each other but are separated by barrier layer (BS). The particular advantage of this configuration is that the—often very thin—barrier layer (BS) is located neither on the inner nor on the outer surface of the multi-layer film (F), but is protected towards the inside by the polymeric layer (P1), and towards the outside by the polymeric layer (P2). In this configuration, mechanical abrasion or mechanical destruction of barrier layer (BS) can thus be avoided as far as possible. It is therefore advantageous in the context of the present disclosure if the at least one multi-layer film (F) contains the at least one barrier layer (BS) between the at least one first polymer layer (P1) and the at least one second polymer layer (P2). The use of such packaging has proven to be particularly advantageous in terms of increased storage stability, as this configuration does not exhibit puffing or delamination on prolonged contact with an oxidizing agent-containing preparation. In another preferred embodiment, barrier layer (BS) is also located between the two polymer layers P1 and P2, but the first polymer layer (P1) is located on the outside of the packaging.

As contemplated herein, the outside of the packaging (VP) is understood to be that side of the packaging which does not come into contact with the cosmetic composition (KM) but with the environment. The use of such packaging has proven to be particularly advantageous in terms of increased storage stability, since this configuration shows neither puffing nor delamination during prolonged contact with an oxidizing agent-containing preparation.

As contemplated herein, the first polymeric material of the first layer (P1) is a polyester. This material may be a layer of one type of polymer or a layer of a polymer mixture. According to the present disclosure, the at least one first polymer layer (P1) is formed from polyethylene terephthalate or polyethylene naphthalate, in particular from polyethylene terephthalate. As contemplated herein, the term “formed” is understood to mean that the polymer layer contains at least about 70% by weight, preferably at least about 80% by weight, more preferably at least about 90% by weight, in particular at least about 99% by weight, in each case based on the total weight of the polymer layer (P1) of the aforementioned compounds.

Polyethylene terephthalate (PET) is a polymer from the group of polyesters. Polyethylene terephthalate may be produced, for example, by transesterification of dimethyl terephthalate with ethylene glycol at higher temperatures. In this transesterification reaction, methanol is split off, which is removed by distillation. The resulting bis(2-hydroxyethyl)-terephthalate is converted to PET by polycondensation, again producing ethylene glycol. Another method of producing polyethylene terephthalate is the direct polycondensation of ethylene glycol and terephthalic acid at high temperatures with distillation of the resulting water. Polyethylene terephthalate is has a particularly high mechanical strength. If the PET layer forms the outer layer, this also offers the advantage that the layer underneath may be printed without the print being rubbed off. The PET layer is transparent and provides a mechanical protective layer for the printed layer.

According to a preferred embodiment of the present disclosure, the layer thickness of the first polymer layer (P1) is from about 4 μm to about 50 μm, preferably from about 5 μm to about 35 μm, more preferably from about 6 μm to about 20 μm. The layer thickness of the PET layer used according to the preferred embodiment is associated with special advantages, which are related to general properties of PET. PET is has a high dimensional stability/stiffness. If PET with these thicknesses is chosen as the first polymer layer (P1), this offers an advantageous mechanical dimensional stability for the film. At the same time, the overall thickness of the film may be kept minimal, so that a material and resource-saving film may be provided.

Furthermore, the multi-layer film (F) from which the packaging is manufactured, comprises a second polymer layer (P2) of a second polymeric material. The second polymeric material may be a layer of one polymer type or a layer of a polymer mixture. It is intended in the context of the present disclosure that the at least one second polymer layer (P2) is formed of a polyolefin, in particular of polyethylene. As contemplated herein, the term “formed” is understood to mean that the polymer layer contains at least about 70% by weight, preferably at least about 80% by weight, more preferably at least about 90% by weight, in particular at least about 99% by weight, in each case based on the total weight of the polymer layer (P2) of the above-mentioned compounds.

The second polymeric material of the second layer (P2) of the multi-layer film (F) is a polyolefin, in particular polyethylene. Polyolefins are polymers which are produced from alkenes such as ethylene, propylene, 1-butene or isobutene by chain polymerization. Polyolefins are saturated hydrocarbons. They are semi-crystalline thermoplastics which are easy to process. They are exemplified by good chemical resistance. Polyethylene and polypropylene are widely used in film applications. As contemplated herein, polypropylene is therefore used for the second layer (P2), but polyethylene is preferred. Polyethylene is produced by polymerization of ethylene using various catalysts. For example, polyethylene may be produced by polymerizing ethylene in the gas phase or in suspension. The average relative molar mass may be controlled, for example, by setting a certain hydrogen partial pressure during the polymerization of ethylene. The processing of polyethylene may be carried out, for example, by extrusion and stretch blow molding, or by pressing, calendering, thermoforming and cold forming.

The second polymer layer (P2) serves as a support layer. Although polyethylene has the disadvantage of being permeable to oxygen and water vapor, it has the advantage of being inexpensive and, due to its low melting point—lower than that of polypropylene—it is easy and energy-saving to process.

According to a preferred embodiment of the present disclosure, the second polymer layer (P2) has a certain layer thickness. According to the preferred embodiment of the present disclosure, the second polymer layer (P2) has a layer thickness of from about 20 μm to about 150 μm, preferably of from about 30 μm to about 110 μm, more preferably of from about 40 μm to about 90 μm. The second polymer layer (P2) has a higher layer thickness than the first polymer layer (P1).

The polymer layers (P1) and (P2) of the multi-layer film (F) comprise organic polymeric materials, which usually have only an insufficient barrier effect against gases and water vapor. If the oxidizing agent-containing composition (KM) is packaged in a packaging (VP) made of a multi-layer film (F), which only comprises the two organic polymer layers (P1) and (P2), water vapor may escape unhindered, so that the water content in the composition (KM) changes in an unacceptable way during prolonged storage. To specifically minimize the uncontrolled escape of water vapor from the packaging (VP), the organic polymer layers (P1) and (P2) are therefore used in combination with a barrier layer (BS).

The barrier layer (BS) functions as a barrier to the passage of gases and water vapor. As contemplated herein, this means that the barrier layer (BS) reduces and controls the permeation rate of water vapor and gases through the film. A film (F) as contemplated herein, which comprises a barrier layer (BS) in addition to the layers (P1) and (P2), thus shows a reduced water vapor permeability and a reduced gas permeability compared to a comparable film (with the same overall thickness), which only comprises the two layers (P1) and (P2), but no barrier layer (BS).

For example, the barrier layer (BS) is a thin layer comprising an inorganic material, which may be applied to organic polymer layers using vacuum coating technologies (e.g. PVD “physical vapour deposition” or CVD “chemical vapour deposition”).

If the barrier layer (BS) is a layer which comprises at least one inorganic material, metals, semimetals or oxides of metals or semimetals, for example aluminum, aluminum oxides, magnesium, magnesium oxides, silicon, silicon oxides, titanium, titanium oxides, tin, tin oxides, zirconium, zirconium oxide and/or carbon may be used for films.

As contemplated herein, the barrier layer (BS) is formed from a polyester provided with an SiOₓ layer, in particular from a polyethylene terephthalate provided with an SiOₓ layer. The SiOₓ layer is particularly well compatible with the adjacent polyethylene layer.

According to a particularly preferred embodiment of the present disclosure, the polyethylene terephthalate layer of the barrier layer (BS) is part of the first layer (P1). In other words, the SiOₓ is vapor-deposited on the first layer (P1). The layer thickness of the PET layer then corresponds to the layer thickness of the PET layer which is part of the barrier layer (BS) and the layer thickness which is part of the first layer (P1).

The polyethylene terephthalate film is provided with a SiOₓ layer. The silicon oxide is vapor-deposited on the polyethylene terephthalate film and is primarily responsible for the barrier effect. According to a preferred embodiment of the present disclosure, the ratio of the layer thickness of SiOₓ to polyethylene terephthalate is from about 1:1000 to about 1:10, preferably from about 1:500 to about 1:50, more preferably from about 1:200 to about 1:100.

The production of films with barrier layers comprising inorganic material is well known. Even the multi-layer film (F) used as contemplated herein may be produced by a process that is used for the manufacture of known films with barrier layers in the state of the art, as described in the documents EP 1036813 A1, EP 2371539 A1 and EP 1541340 A1.

The barrier layer (BS) may also include a thin layer of inorganic-organic hybrid polymers. These polymers are known in the literature under the technical term ORMOCER polymers. A typical ORMOCER polymer may be produced, for example, by hydrolytic polycondensation of an organofunctional silane with an aluminum compound and possibly with an inorganic oxide component. Corresponding syntheses are disclosed in document EP 0792846 B 1, which is referred to in full here. Inorganic-organic hybrid polymers (ORMOCER polymers) have both inorganic and organic network structures. The inorganic silicate network structure may be formed in the sol-gel process by controlled hydrolysis and condensation of alkoxy silanes. The silicate network may be specifically modified by including metal alkoxides in the sol-gel process. By polymerization of organofunctional groups, which are introduced into the material by the organoalkoxysilanes, an additional organic network is built up. The ORMOCER polymers produced in this way may be applied to the layers (P1) and/or (P2) using conventional application technologies (spraying, brushing, etc.).

The thicker the barrier layer (BS), the greater or stronger is the barrier effect against the passage of gases and water vapor. The thickness of the barrier layer (BS) may therefore be selected depending on the desired barrier effect. According to a preferred embodiment of the present disclosure, the at least one barrier layer (BS) has a layer thickness of from about 4 μm to about 20 μm, preferably of from about 5 μm to about 18 μm, more preferably of from about 6 μm to about 15 μm.

The material, the structure and the layer thicknesses determine the permeability values of the film. The multi-layer film (F) of the packaging of the cosmetic product as contemplated herein has advantageous properties with regard to oxygen permeability and water vapor permeability. The multi-layer film exhibits an oxygen transmission rate (OTR) at 23° C. and 50% relative humidity of from about 0.1 to about 5 cc/m²/d/bar, preferably of from about 0.2 to about 3.5 cc/m²/d/bar, more preferably of from about 0.5 to about 2.5 cc/m²/d/bar, and a water vapor transmission rate at 38° C. and 100% relative humidity of from about 0.1 to about 5 g/m²d, preferably of from about 0.2 to about 3.5 g/m²d, more preferably of from about 0.5 to about 2.5 g/m²d.

As contemplated herein, the permeability values of the film (F) are advantageously adjusted. The film (F) thus provides the packaging with advantageous barrier properties, especially with respect to the water vapor transmission rate (WVTR) measured in g/(m²d) or g/(m² 24 h) units as determined by the ASTM F 1249 method at 38° C. ambient temperature and 100% relative humidity, and for oxygen; (OTR, measured in cm³/(m²d bar) or cm³/(m² 24 h)), where cm³ is equal to cc, at an atmospheric pressure of 1 bar) measured according to ASTM D 3985 at 23° C. ambient temperature and 50% relative humidity.

In addition to layers (P1), (P2) and (BS) described so far, the multi-layer film (F) may also comprise one or more further layers. These additional layers may be, for example, intermediate layers and/or adhesive layers. As contemplated herein, it is therefore preferred if the at least one multi-layer film (F) additionally contains at least one further layer selected from the group of intermediate layers (SZ), adhesive layers (SK) and mixtures thereof.

For example, films (F) may have further intermediate layers (SZ) to increase the mechanical stability. Intermediate layers may also prevent or minimize the permeation of polymers or remaining monomers from a polymer layer into the cosmetic composition (KM).

To increase bond strength, the films may also include one or more adhesive layers (SK) to reduce or prevent delamination (i.e. peeling or formation of an air space) between two layers.

A particularly preferred product as contemplated herein is exemplified in that the multi-layer film (F) comprises, in addition to the first polymer layer (P1), the second polymer layer (P2) and the barrier layer (BS), one or more further layers selected from intermediate layers (SZ) and/or adhesive layers (SK).

If the multi-layer film (F) contains other layers in addition to the layers (P1), (P2) and (BS), the following configurations of the layers are possible (from the interior (in contact with the cosmetic composition (KM)) to the exterior):
a) *interior*-layer (P1)-first adhesive layer (SK1)-layer (P2)-second adhesive layer (SK2)-barrier layer (BS)-*exterior*,
b) *interior*-layer (P1)-adhesive layer (SK1)-layer (P2)-barrier layer (BS)-*exterior*,
c) *interior*-layer (P1)-layer (P2)-second adhesive layer (SK2)-barrier layer (BS)-*exterior*,
d) *interior*-barrier layer (BS)-first adhesive layer (SK1)-layer (P1)-second adhesive layer (SK2)-layer (P2)-*exterior*,
e) *iinterior*-barrier layer (BS)-adhesive layer (SK)-layer (P1)-layer (P2)-*exterior*,
f) *interior*-barrier layer (BS)-layer (Si)-adhesive layer (SK)-layer (P2)-*exterior*,
g) *interior*-layer (P1)-first adhesive layer (SK1)-barrier layer (BS)-second adhesive layer (SK2)-layer (P2)-*exterior*,
h) *interior*-layer (P1)-adhesive layer (SK)-barrier layer (BS)-layer (P2)-*exterior*,
i) *interior*-layer (P1)-barrier layer (BS)-adhesive layer (SK)-layer (P2)-*exterior*.

In any case, the film should be designed so that there is sufficient adhesion between the films. According to a preferred embodiment of the present disclosure, the bond strength of the film is from about 0.1 to about 10 N/15 mm, preferably from about 1 to about 8 N/15 mm, more preferably from about 1.5 to about 5 N/15 mm. This is measured according to ASTM F-904. Bond strength is a physical measure of the adhesive force between the layers. It is related to the two layers of a film with the lowest bond strength between two layers of the same film. The adhesive strengths set as contemplated herein result in a favorable mechanical stability over the storage time of the packaged cosmetic product.

Furthermore, the strength between two bonded (sealed or sealed-off) films should be sufficient. According to a preferred embodiment of the present disclosure, the seal strength of the packaging (VP) is from about 10 to about 40 N/15 mm, preferably from about 15 to about 35 N/15 mm, more preferably from about 20 to about 30 N/15 mm, under the conditions 150° C., 2.54 cm (1″) and 4 kg/cm². The seal strength is measured according to ASTM F-88 under the above conditions. The challenge with packaging is always to ensure the mechanical durability of the packaging while at the same time making the contents easily accessible to the user. Adjusting the seal strength to these values enables both of these objectives to be achieved.

A sealed seam is a seam by which the packaging is closed. Usually, two films are placed on top of each other for closing the package and are pressed together by a force perpendicular to the film surface. By heating the films in the area that is being compressed, parts of the compressed areas can fuse together so that the films are welded together. There may also be an adhesive between the compressed films that strengthens the seam.

The product as contemplated herein comprises as a second component a cosmetic composition (KM) which is packaged in the packaging (VP) and which comprises at least one oxidizing agent, a linear C₈-C₃₀ alcohol and a non-ionic surfactant.

The intended use of the product as contemplated herein is oxidative color change. For this purpose—as already described above—a cosmetic composition (KM) containing an oxidizing agent is usually mixed with a second preparation (B) which is packaged separately from (KM). In this way, the ready-to-use oxidative color changing agent is produced. Depending on whether the oxidative color change is intended to achieve a bleaching, lightening or coloring effect, preparation (B) may contain different ingredients. If a mere lightening or bleaching is desired, preparation (B) contains at least one alkalizing agent. If oxidative dyeing is desired, preparation (B) often contains oxidation dye precursors in addition to the alkalizing agent. In order to ensure that the preparations (KM) and (B) can be mixed sufficiently quickly, both the preparation (KM) and the preparation (B) are usually free-flowing, aqueous or water-containing preparations.

As contemplated herein, preparation (KM) is an aqueous preparation. The water content of preparation (KM) may be—based on the total weight of the preparation (KM)—for example from about 60 to about 97% by weight, preferably from about 75 to about 93% by weight, more preferably from about 78 to about 91% by weight, in particular from about 80 to about 88.0% by weight. All percentages by weight refer to the total weight of water contained in the preparation (KM), which is related to the total weight of the preparation (KM).

The cosmetic composition (KM) contains as the first essential ingredient a) at least one oxidizing agent. Preference is given to certain oxidizing agents. It is therefore advantageous in the context of the present disclosure if the cosmetic composition (KM) contains at least one oxidizing compound selected from the group including persulfates, chlorites, hydrogen peroxide and attachment products of hydrogen peroxide to urea, melamine and sodium borate, in particular hydrogen peroxide. As contemplated herein, the use of hydrogen peroxide has proven to be particularly advantageous.

The concentration of the oxidizing agent in the composition (KM) is determined on the one hand by the legal requirements and on the other hand by the desired effect; preferably from about 0.5 to about 20.0% by weight solutions in water are used. It is therefore preferred as contemplated herein if the cosmetic composition (KM) contains at least one oxidizing compound, in particular hydrogen peroxide, in a total amount of from about 0.5 to about 20% by weight, preferably from about 1.0 to about 18% by weight, more preferably from about 1.2 to about 16% by weight, in particular from about 1.5 to about 15% by weight, based on the total weight of the cosmetic composition (KM). The higher the content of oxidizing agent, in particular hydrogen peroxide, in the composition (KM), the greater is the amount of gas produced in the case of a proportional decomposition of the oxidizing agent. Preparations containing a higher concentration of oxidizing agent are therefore much more difficult to store in a packaging (VP) than less concentrated preparations. The amount of hydrogen peroxide refers to 100% hydrogen peroxide.

In the course of the work leading to this present disclosure, it was found that the product as contemplated herein is particularly suitable for packaging and stable storage of higher concentrated hydrogen peroxide preparations (KM). Thus, packaging (VP) as contemplated herein containing preparations (KM) with from about 9 to about 12% by weight hydrogen peroxide did not show any changes in volume (i.e. no swelling) and no unplanned openings (i.e. the packaging did not burst open) even after storage at elevated temperature for several weeks.

The cosmetic composition (KM) contains as the second essential ingredient b) at least one C₈-C₃₀ alcohol. In this context mixtures of linear C₁₆-C₁₈ alcohols have proved particularly useful. Such mixtures in combination with the further feature c) of the composition (KM) lead to an excellent stabilization of the at least one oxidizing agent, in particular of hydrogen peroxide. It is therefore advantageous in the context of the present disclosure if the cosmetic composition (KM) contains at least one C10-C₃₀ alcohol selected from the group of linear C10-C₂₈ alcohols, linear C₁₂-C₂₆ alcohols, linear C₁₄-C₂₀ alcohols, linear C₁₄-C₁₈ alcohols as well as mixtures of the aforementioned alcohols, in particular a mixture of linear C₁₆-C₁₈ alcohols. In the context of the present disclosure, the mixture of cetyl alcohol and stearyl alcohol known as cetearyl alcohol, in particular a mixture of about 50% by weight of cetyl alcohol and about 50% by weight of stearyl alcohol, based on the total weight of the mixture, has proved to be particularly advantageous.

The at least one C₈-C₃₀ alcohol is preferably used in certain quantity ranges. Preferred embodiments of the present disclosure are exemplified in that the cosmetic composition (KM) contains the at least one C₈-C₃₀ alcohol, in particular a mixture of linear C₁₆-C₁₈ alcohols, in a total amount of from about 0.1 to about 10% by weight, preferably of from about 0.50 to about 6.5% by weight, more preferably of from about 1.0 to about 6.0% by weight, in particular of from about 1.5 to about 5.0% by weight, based on the total weight of the cosmetic composition (KM). The use of the total amounts of at least one C₈-C₃₀ alcohol, in particular the mixture of linear C₁₆-C₁₈ alcohols, as indicated above, in combination with the other ingredients of the cosmetic composition (KM) leads to a particularly good stabilization of the oxidizing agent contained in this composition, in particular hydrogen peroxide.

As third essential ingredient c), the cosmetic composition (KM) contains at least one non-ionic surfactant. The term “non-ionic surfactant” is understood to mean, as contemplated herein, amphiphilic (bifunctional) compounds having at least one hydrophobic and at least one hydrophilic part of the molecule. The hydrophobic portion is preferably a hydrocarbon chain with from about 8 to about 28 carbon atoms, which can be saturated or unsaturated, linear or branched. This C₈-C₂₈ alkyl chain is particularly preferred linear. In contrast to anionic, cationic, zwitterionic and amphiphilic surfactants, however, non-ionic surfactants contain neither cationic nor anionic groups. Furthermore, these surfactants also do not contain any cationizable and anionizable groups which may form cationic or anionic groups depending on the pH value.

The combination of fatty alcohol and non-ionic surfactant ensures excellent dispersion of the components of the cosmetic composition (KM) and thus a high storage stability. In addition, the use of such a combination leads to a good spreadability, especially miscibility, of the cosmetic composition (KM) with the preparation (B) containing the oxidation dye precursors. It is therefore preferred in the context of the present disclosure if the cosmetic composition (KM) contains at least one non-ionic surfactant selected from the group including (i) ethoxylated and/or propoxylated alcohols and carboxylic acids having from about 8 to about 30 carbon atoms and from about 2 to about 30 ethylene oxide and/or propylene oxide units per mol of alcohol, (ii) attachment products of from about 30 to about 50 mol of ethylene oxide to castor oil and hydrogenated castor oil, (iii) alkyl polyglucosides corresponding to the formula R¹O-[G]ₚ, in which R1 is an alkyl and/or alkenyl portion containing from about 4 to about 22 carbon atoms, G is a sugar portion containing 5 or 6 carbon atoms and p is a number of from about 1 to about 10, (iv) monoethanolamides of carboxylic acids containing from about 8 to about 30 carbon atoms and (v) mixtures thereof, more particularly attachment products of 40 mol ethylene oxide to hydrogenated castor oil. In the formula R¹O-[G]ₚ, the index number p indicates the degree of oligomerization (DP), i.e. the distribution of mono- and oligoglucosides, and represents a number between about 1 and about 10. While p in a given compound must always be an integer and can assume the values p=from about 1 to about 6, the value p for a specific alkyl oligoglucoside is an analytically determined mathematical value, which is usually a fractional number. As contemplated herein, alkyl and/or alkenyl oligoglucosides with an average degree of oligomerization p of from about 1.1 to about 3.0 are preferably used. From the point of view of application technology, those alkyl and/or alkenyl oligoglucosides are preferred whose degree of oligomerization is less than about 1.7 and in particular lies between about 1.2 and about 1.7. The alkyl or alkenyl radical R¹ may be derived from primary alcohols containing from about 4 to about 20, preferably from about 8 to about 16 carbon atoms. As contemplated herein, alkyl oligoglucosides based on hardened C_{12/14} coconut alcohol with a DP of from about 1-3, as they are commercially available under the INCI designation “coco-glucosides”, for example, are particularly preferred. The non-ionic surfactants used with particular preference in the context of the present disclosure are attachment products of 40 mol ethylene oxide to hydrogenated castor oil, in particular the compound known under the INCI designation PEG-40 hydrogenated castor oil (CAS no.: 61788-85-0).

To ensure sufficient dispersion of all ingredients of the cosmetic product (KM), at least one non-ionic surfactant is preferably used in certain total quantities. Preferential embodiments of the present disclosure are exemplified in that the cosmetic composition (KM) contains the at least one non-ionic surfactant, in particular the mixture of linear C₁₆-C₁₈ alcohols substituted with an average of 20 ethoxy groups, in a total amount of from about 0.10 to about 5% by weight of the total amount of KM, preferably from about 0.12 to about 3.5% by weight, more preferably from about 0.15 to about 1.8% by weight, in particular from about 0.50 to about 1.5% by weight, based on the total weight of the cosmetic composition (KM).

The cosmetic composition (KM) may include at least one liquid cosmetic oil as a further ingredient. In the context of the present disclosure, the term “liquid cosmetic oils” means oils suitable for cosmetic use which are insoluble in water at 20° C., i.e. preferably less than 1% by weight of the oil, based on the total amount of the water/oil mixture, dissolve in water at 20° C. However, the cosmetic oil used as contemplated herein is neither a perfume nor an essential oil. Preferably paraffin oils are used as cosmetic oils within the context of the present disclosure. The use of cosmetic oils leads to an increased stabilization of the oxidizing agent, in particular of the hydrogen peroxide, as the latter is surrounded by the cosmetic oil when dispersed or emulsified with the cosmetic oil and thus protected against decomposition due to the reaction with further components of the cosmetic composition (KM). In the context of the present disclosure, certain cosmetic oils are preferably used. As contemplated herein, it is therefore advantageous if the cosmetic composition (KM) contains at least one liquid cosmetic oil selected from the group of (i) esters of linear or branched saturated or unsaturated C₂-C₃₀ fatty alcohols with linear or branched saturated or unsaturated C₂-C₃₀ fatty acids, which may be hydroxylated, (ii) C₈-C₂₂ fatty alcohol esters of mono- or polyhydric C₂-C₇ hydroxycarboxylic acids, the triethyl citrates, (iii) mono-, di- and triglycerides of linear or branched, saturated or unsaturated, optionally hydroxylated C₈-C₃₀ fatty acids, (iv) dicarboxylic acid esters of linear or branched C₂-C₁₀ alkanols, (v) symmetrical, asymmetrical or cyclic esters of carbonic acid with fatty alcohols, the esters of dimers of unsaturated C₁₂ -C₂₂ fatty acids with monovalent, linear, branched and cyclic C₂-C₁₈ alkanols or C₂-C₆ alkanols (vi) benzoic acid esters of linear or branched C₈-C₂₂ alkanols, such as benzoic acid C₁₂-C₁₅ alkyl esters, benzoic acid isostearyl esters and benzoic acid octyldodecyl esters, (vii) synthetic hydrocarbons, such as polyisobutene and polydecenes, (viii) hydrocarbons, such as paraffins, and (ix) mixtures thereof, in particular paraffin oil. In the context of the present disclosure, it has been found to be particularly advantageous to use paraffin oil, in particular the compound known under the INCI designation Paraffinum Liquidqum (CAS no.: 8042-47-5), as liquid cosmetic oil. Paraffin oils preferred as contemplated herein have dynamic viscosities at 20° C. of from about 20 to about 150 mPa*s (measured according to DIN 51562-1 of 1999).

The at least one liquid cosmetic oil, in particular the at least one paraffin oil, is contained in the cosmetic compositions (KM) preferably in certain total amounts. This ensures sufficient stabilization of the oxidizing agent, in particular hydrogen peroxide. Preferred embodiments of the present disclosure are exemplified in that the cosmetic composition (KM) contains the at least one liquid cosmetic oil, in particular paraffin oil, in a total amount of from about 0.10 to about 25% by weight, preferably of from about 2.0 to about 24% by weight, more preferably of from about 4.0 to about 22% by weight, in particular from about 5.0 to about 20% by weight, based on the total weight of the cosmetic composition (KM).

In the course of the work leading to this present disclosure, it has been found that the use of the aforementioned essential ingredients b) and c) ensures that the cosmetic composition (KM) containing at least one oxidizing agent may be packaged and stored in the special packaging (VP) without this packaging—which has a barrier layer with a passage barrier effect for gases and water vapor—swelling or bursting.

In this context, a very specific combination of ingredients a) to c) of the cosmetic composition (KM) has proven to be advantageous. In a preferred embodiment, the product as contemplated herein is exemplified in that the cosmetic composition (KM) comprises hydrogen peroxide, a mixture of linear C₁₆-C₁₈ alcohols and, as a non-ionic surfactant, a mixture of linear C₁₆-C₁₈ alcohols substituted with an average of 20 ethoxy groups.

For further optimization of storage stability, the above-mentioned compounds are preferably used in certain quantity ranges in the preparation (KM). Particularly preferred embodiments are exemplified in that the cosmetic composition (KM) contains
a) from about 1.5 to about 15% by weight of hydrogen peroxide,
b) from about 1 to about 5% by weight of a mixture of linear C₁₆-C₁₈ alcohols, and
c) from about 0.5 to about 3.5% by weight of a mixture of linear C₁₆-C₁₈ alcohols which are substituted with an average of 20 ethoxy groups, each based on the total weight of the cosmetic product (KM).

The cosmetic composition of the present disclosure may additionally contain at least one or more cationic surfactants. As contemplated herein, the term “cationic surfactant” means amphiphilic (bifunctional) compounds includes at least one hydrophobic and at least one hydrophilic part of the molecule. The hydrophobic portion is preferably a hydrocarbon chain with from about 8 to about 28 carbon atoms, which may be saturated or unsaturated, linear or branched. This C₈-C₂₈ alkyl chain is particularly preferred linear. In addition, these surfactants contain at least one cationic group, in particular an ammonium or alkylammonium group. Quaternary alkylammonium compounds, in particular tetraalkylammonium compounds with at least one C₁₂-C₂₀ alkyl group, with a physiologically compatible anion, in particular a chloride, are preferred.

Typical examples of these ingredients are cetyl trimethyl ammonium chloride, steartrimonium chloride, behentrimonium chloride, stearamidopropyl trimonium chloride, dioleylethyl dimethyl ammonium methosulfate and dioleylethyl hydroxyethylmonium methosulfate, especially steartrimonium chloride.

The at least one cationic surfactant, in particular steartrimonium chloride, is present in the cosmetic composition (KM) preferably in certain total amounts. This ensures that the ready-to-use colorant may be applied to the hair in stable dispersion. Preferential embodiments of the present disclosure are exemplified in that the cosmetic composition (KM) contains the at least one cationic surfactant in an amount of from about 0.01 to about 2% by weight, preferably in an amount of from about 0.05 to about 1% by weight, more preferably in an amount of from about 0.1 to about 0.5% by weight, each based on the total weight of the cosmetic composition.

The cosmetic composition (KM) preferably has an acidic pH value in order to avoid or reduce decomposition of the oxidizing agent used, in particular hydrogen peroxide. It is therefore preferred within the context of the present disclosure that the cosmetic composition (KM) has a pH value (measured at 20° C.) of from about pH 1.5 to about pH 5.0, preferably of from about pH 2.0 to about pH 4.7, more preferably of from about pH 2.3 to about pH 4.4, in particular of from about pH 2.5 to about pH 4.

The preparation (KM) contained in the packaging (VP) contains the essential ingredients in an aqueous or aqueous-alcoholic carrier, which may be a cream, an emulsion, a gel or a surfactant-containing foaming solution. To adjust the desired properties of these dosage forms, the preparation (KM) may also contain additional active ingredients, auxiliary substances and additives.

The preparation (KM) may also contain one or more acids to stabilize the oxidizing agent used, especially hydrogen peroxide. It is therefore preferred within the context of the present disclosure if the cosmetic composition (KM) additionally contains at least one acid selected from the group of dipicolinic acid, citric acid, acetic acid, malic acid, lactic acid, tartaric acid, hydrochloric acid, phosphoric acid, pyrophosphoric acid and their salts, benzoic acid and its salts, 1-hydroxyethane-1,1-diphosphonic acid, ethylenediaminetetraacetic acid and its salts, sulphuric acid and mixtures, in particular a mixture of dipicolinic acid, disodium pyrophosphate, benzoic acid and its salts and 1-hydroxyethane-1,1-diphosphonic acid.

A particularly high stabilization of the oxidizing agent, especially of the hydrogen peroxide, is achieved when the above-mentioned acids are used in certain quantity ranges. It is therefore advantageous in this context if the at least one acid, in particular the mixture of dipicolinic acid, disodium pyrophosphate, benzoic acid and its salts and 1-hydroxyethane-1,1-diphosphonic acid, is used in a total amount of from about 0.1 to about 3.0% by weight, preferably from about 0.5 to about 2.5% by weight, more preferably from about 0.8 to about 2.0% by weight, in particular from about 0.9 to about 1.5% by weight, based on the total weight of the cosmetic composition (KM).

The following tables list the most preferred embodiments AF 1 to AF 32 of the cosmetic composition (KM) contained in the packaging (VP) (all figures in percentage by weight unless otherwise indicated).

| | AF 1 | AF 2 | AF 3 | AF 4 |
|---|---|---|---|---|
| Oxidizing agent | 0.5-20 | 1.0-18 | 1.2-16 | 1.5-15 |
| C₈-C₃₀ alcohol | 0.10-7.0 | 0.50-6.5 | 1.0-6.0 | 1.5-5.0 |
| Non-ionic surfactant | 0.10-2.5 | 0.12-2.0 | 0.15-1.8 | 0.30-1.5 |
| Cosmetic carrier ¹⁾ | ad 100 | ad 100 | ad 100 | ad 100 |

| | AF 5 | AF 6 | AF 7 | AF 8 |
|---|---|---|---|---|
| Oxidizing agent ²⁾ | 0.5-20 | 1.0-18 | 1.2-16 | 1.5-15 |
| C₈-C₃₀ alcohol | 0.10-7.0 | 0.50-6.5 | 1.0-6.0 | 1.5-5.0 |
| Non-ionic surfactant | 0.10-2.5 | 0.12-2.0 | 0.15-1.8 | 0.30-1.5 |
| Cosmetic carrier ¹⁾ | ad 100 | ad 100 | ad 100 | ad 100 |

| | AF 9 | AF 10 | AF 11 | AF 12 |
|---|---|---|---|---|
| Oxidizing agent ²⁾ | 0.5-20 | 1.0-18 | 1.2-16 | 1.5-15 |
| C₈-C₃₀ alcohol ³⁾ | 0.10-7.0 | 0.50-6.5 | 1.0-6.0 | 1.5-5.0 |
| Non-ionic surfactant | 0.10-2.5 | 0.12-2.0 | 0.15-1.8 | 0.30-1.5 |
| Cosmetic carrier ¹⁾ | ad 100 | ad 100 | ad 100 | ad 100 |

| | AF 13 | AF 14 | AF 15 | AF 16 |
|---|---|---|---|---|
| Oxidizing agent ²⁾ | 0.5-20 | 1.0-18 | 1.2-16 | 1.5-15 |
| C₈-C₃₀ alcohol ³⁾ | 0.10-7.0 | 0.50-6.5 | 1.0-6.0 | 1.5-5.0 |
| Non-ionic surfactant | 0.10-2.5 | 0.12-2.0 | 0.15-1.8 | 0.30-1.5 |
| Liquid cosmetic oil | 0.10-25 | 2.0-24 | 4.0-22 | 5.0-20 |
| Cosmetic carrier ¹⁾ | ad 100 | ad 100 | ad 100 | ad 100 |

| | AF 17 | AF 18 | AF 19 | AF 20 |
|---|---|---|---|---|
| Oxidizing agent ²⁾ | 0.5-20 | 1.0-18 | 1.2-16 | 1.5-15 |
| C₈-C₃₀ alcohol ³⁾ | 0.10-7.0 | 0.50-6.5 | 1.0-6.0 | 1.5-5.0 |
| Non-ionic surfactant ⁵⁾ | 0.10-2.5 | 0.12-2.0 | 0.15-1.8 | 0.30-1.5 |
| Liquid cosmetic oil | 0.10-25 | 2.0-24 | 4.0-22 | 5.0-20 |
| Cosmetic carrier ¹⁾ | ad 100 | ad 100 | ad 100 | ad 100 |

| | AF 21 | AF 22 | AF 23 | AF 24 |
|---|---|---|---|---|
| Oxidizing agent ²⁾ | 0.5-20 | 1.0-18 | 1.2-16 | 1.5-15 |
| C₈-C₃₀ alcohol ³⁾ | 0.10-7.0 | 0.50-6.5 | 1.0-6.0 | 1.5-5.0 |
| Non-ionic surfactant ⁵⁾ | 0.10-2.5 | 0.12-2.0 | 0.15-1.8 | 0.30-1.5 |
| Cosmetic carrier ¹⁾ | ad 100 | ad 100 | ad 100 | ad 100 |

| | AF 25 | AF 26 | AF 27 | AF 28 |
|---|---|---|---|---|
| Oxidizing agent ²⁾ | 0.5-20 | 1.0-18 | 1.2-16 | 1.5-15 |
| C₈-C₃₀ alcohol ³⁾ | 0.10-7.0 | 0.50-6.5 | 1.0-6.0 | 1.5-5.0 |
| Cationic surfactant ⁴⁾ | 0.10-3.0 | 0.12-2.5 | 0.15-2.0 | 0.20-1.5 |
| Non-ionic surfactant ⁵⁾ | 0.10-2.5 | 0.12-2.0 | 0.15-1.8 | 0.30-1.5 |
| Liquid cosmetic oil ⁶⁾ | 0.10-25 | 2.0-24 | 4.0-22 | 5.0-20 |
| Acid | 0.1-3.0 | 0.5-2.5 | 0.8-2.0 | 0.9-1.5 |
| Cosmetic carrier ¹⁾ | ad 100 | ad 100 | ad 100 | ad 100 |

| | AF 29 | AF 30 | AF 31 | AF 32 |
|---|---|---|---|---|
| Oxidizing agent ²⁾ | 0.5-20 | 1.0-18 | 1.2-16 | 1.5-15 |
| C₈-C₃₀ alcohol ³⁾ | 0.10-7.0 | 0.50-6.5 | 1.0-6.0 | 1.5-5.0 |
| Cationic surfactant ⁴⁾ | 0.10-3.0 | 0.12-2.5 | 0.15-2.0 | 0.20-1.5 |
| Non-ionic surfactant ⁵⁾ | 0.10-2.5 | 0.12-2.0 | 0.15-1.8 | 0.30-1.5 |
| Acid ⁷⁾ | 0.1-3.0 | 0.5-2.5 | 0.8-2.0 | 0.9-1.5 |
| Cosmetic carrier ¹⁾ | ad 100 | ad 100 | ad 100 | ad 100 |

1) Aqueous or aqueous-alcoholic carrier
2) Hydrogen peroxide, amount calculated on 100% hydrogen peroxide
3) Mixture of linear C₁₆-C₁₈ alcohols, especially cetearyl alcohol
4) Quaternary ammonium compounds, especially steartrimonium chloride
5) Mixture of linear C₁₆-C₁₈ alcohols substituted with an average of 20 ethoxy groups
6) Paraffin oil
7) Mixture of dipicolinic acid, disodium pyrophosphate, benzoic acid and its salts and 1-hydroxyethane-1,1-diphosphonic acid

The embodiments AF 1 to 32 described above are each packaged in packaging (VP) which has the configuration of the multi-layer film (F) described below (viewed from the interior (in contact with the cosmetic composition (KM)) to the exterior):
a) *interior*-layer (P1)-layer (P2)-barrier layer (BS)-*exterior*,
b) *interior*-layer (P1)-barrier layer (BS)-layer (P2)-*exterior*,
c) *interior*-layer (P2)-layer (P1)-barrier layer (BS)-*exterior*,
d) *interior*-layer (P2)-barrier layer (BS)-layer (P1)-*exterior*,
e) *interior*-barrier layer (BS)-layer (P1)-layer (P2)-*exterior*,
f) *interior*-barrier layer (BS)-layer (P2)-layer (P1)-*exterior*,
g) *interior*-layer (P1)-first adhesive layer (SK1)-layer (P2)-second adhesive layer (SK2)-barrier layer (BS)-*exterior*,
h) *interior*-layer (P1)-adhesive layer (SK1)-layer (P2)-barrier layer (BS)-*exterior*,
i) *interior*-layer (P1)-layer (P2)-second adhesive layer (SK2)-barrier layer (BS)-*exterior*,
j) *interior*-barrier layer (BS)-first adhesive layer (SK1)-layer (P1)-second adhesive layer (SK2)-layer (P2)-*exterior*,
k) *interior*-barrier layer (BS)-adhesive layer (SK)-layer (P1)-layer (P2)-*exterior*,
l) *interior*-barrier layer (BS)-layer (Si)-adhesive layer (SK)-layer (P2)-*exterior*,
m) *interior*-layer (P1)-first adhesive layer (SK1)-barrier layer (BS)-second adhesive layer (SK2)-layer (P2)-*exterior*,
n) *interior*-layer (P1)-adhesive layer (SK)-barrier layer (BS)-layer (P2)-*exterior*,
o) *interior*-layer (P1)-barrier layer (BS)-adhesive layer (SK)-layer (P2)-*exterior*.

The products manufactured in this way show high storage stability and water loss within an acceptable range during storage. No swelling or delamination of the packaging (VP) during storage of these cosmetic products as contemplated herein was observed.

The product as contemplated herein is used for the purpose of oxidative color change. For this purpose, the preparation (KM) packaged in the packaging (VP), which is the oxidizing agent preparation, is mixed with at least one other preparation (B) to produce the ready-to-use color changing product. To prevent incompatibilities or premature reaction, the preparations (KM) and (B) are made up separately.

A particularly preferred product as contemplated herein is exemplified in that it comprises a preparation (B) separately packaged from the preparation (KM), the preparation (B) containing at least one compound selected from oxidation dye precursors, direct drawing dyes, alkalizing agents and mixtures thereof. Preferred products of the present disclosure are further exemplified in that they additionally comprise at least one second cosmetic composition (KM2) which contains at least one compound selected from oxidation dye precursors, direct drawing dyes, alkalizing agents and mixtures thereof and which is packaged separately from the cosmetic composition (KM).

If oxidative coloring is desired, the preparation (B) contains at least one oxidation dye precursor. Oxidation dye precursors can be divided into developers and couplers. Due to their greater sensitivity to oxygen, the developers are usually used in the form of their physiologically compatible salts (e.g. in the form of their hydrochlorides, hydrobromides, hydrogen sulphates or sulphates). Coupler components alone do not form a significant coloration in the course of oxidative coloration, but always require the presence of developer components. Preferably such products contain at least one oxidation dye precursor of the developer type and at least one oxidation dye precursor of the coupler type. Particularly suitable oxidation dye precursors of the developer type are selected from at least one compound from the group formed by p-phenylenediamine, p-toluylenediamine, 2-(2-hydroxyethyl)-p-phenylenediamine, 2-(1,2-dihydroxyethyl)-p-phenylenediamine, N,N-bis-(2-hydroxyethyl)-p-phenylenediamine, 2-methoxymethyl-p-phenylenediamine, N-(4-amino-3-methylphenyl)-N-[3 -(1H-imidazol-1-yl)propyl]amine N,N′-bis-(2-hydroxyethyl)-N,N′-bis-(4-aminophenyl)-1,3-diamino-propan-2-ol, bis-(2-hydroxy-5-aminophenyl)methane, 1,3-bis-(2,5-diaminophenoxy)propan-2-ol, N,N′-bis-(4-aminophenyl)-1,4-diazacycloheptane, 1,10-bis-(2,5-diaminophenyl)-1,4,7,10-tetraoxadecane p-aminophenol, 4-amino-3-methylphenol, 4-amino-2-aminomethylphenol, 4-amino-2-(1,2-dihydroxyethyl)phenol, 4-amino-2-(diethylaminomethyl)phenol 4,5-diamino-1-(2-hydroxyethyl)pyrazole, 2,4,5,6-tetraamino-pyrimidine, 4-hydroxy-2,5,6-triaminopyrimidine, 2-hydroxy-4,5,6-triamino-pyrimidine, 2,3-diamino-6,7-dihydro-1H,5H-pyrazolo[1,2-a]pyrazol-1-on and their physiologically compatible salts.

Particularly suitable oxidation dye precursors of the coupler type are selected from the group formed by 3-amino phenol, 5-amino-2-methyl phenol, 3-amino-2-chloro-6-methyl phenol, 2-hydroxy-4-aminophenoxyethanol, 5-amino-4-chloro-2-methylphenol, 5-(2-hydroxyethyl)-amino-2-methylphenol, 2,4-di-chloro-3-aminophenol, 2-aminophenol, 3-phenylene-diamine, 2-(2,4-diaminophenoxy)ethanol, 1,3-bis(2,4-diaminophenoxy)propane, 1-methoxy-2-amino-4-(2-hydroxyethylamino)benzene, 1,3-bis(2,4-diaminophenyl)propane, 2,6-bis(2′-hydroxyethylamino)-1-methylbenzene, 2-({3-[(2-hydroxyethyl)amino]-4-methoxy-5-methylphenyl}amino)ethanol, 2-({3-[(2-hydroxyethyl)amino]-2-methoxy-5-methylphenyl}amino)ethanol, 2-({3-[(2-hydroxyethyl)amino]-4,5-dimethylphenyl}amino)ethanol, 2-[3 -morpholin-4-ylphenyl)amino]ethanol, 3-amino-4-(2-methoxy-ethoxy)-5-methylphenylamine, 1-amino-3-bis-(2-hydroxyethyl)aminobenzene, resorcinol, 2-methylresorcinol, 4-chlororesorcinol, 1,2,4-trihydroxybenzene, 2-amino-3-hydroxypyridine, 3-amino-2-methylamino-6-methoxypyridine, 2,6-dihydroxy-3,4-dimethylpyridine, 3,5-diamino-2,6-dimethoxypyridine, 1-phenyl-3-methylpyrazol-5-on, 1-naphthol, 1,5-dihydroxy-naphthalene, 2,7-dihydroxynaphthalene, 1,7-dihydroxynaphthalene, 1,8-dihydroxy-naphthalene, 4-hydroxy-indole, 6-hydroxyindole, 7-hydroxyindole, 4-hydroxyindoline, 6-hydroxyindoline, 6-hydroxyindoline, 7-hydroxyindoline or mixtures of these compounds or their physiologically compatible salts.

In addition, preparation (B) may also contain one or more direct drawing dyes. Suitable non-ionic direct drawing dyes may be selected from the group HC Yellow 2, HC Yellow 4, HC Yellow 5, HC Yellow 6, HC Yellow 12, HC Orange 1, Disperse Orange 3, HC Red 1, HC Red 3, HC Red 7, HC Red 10, HC Red 11, HC Red 13, HC Red BN, HC Blue 2, HC Blue 11, HC Blue 12, Disperse Blue 3, HC Violet 1, Disperse Violet 1, Disperse Violet 4, Disperse Black 9, 1,4-diamino-2-nitrobenzene, 2-amino-4-nitrophenol, 1,4-bis(2-hydroxyethyl)-amino-2-nitrobenzene, 3 -nitro-4-(2-hydroxyethyl)aminophenol, 2-(2-hydroxyethyl) amino-4,6-dinitrophenol, 4-[(2-hydroxyethyl) amino]-3-nitro-1-methylbenzene, 1-amino-4-(2-hydroxyethyl)amino-5-chloro-2-nitrobenzene, 4-amino-3-nitrophenol, 1-(2′-ureidoethyl)amino-4-nitrobenzene, 2-[(4-amino-2-nitrophenyl)amino]benzoic acid, 4-[(3-hydroxypropyl)amino]-3 -nitrophenol, 4-nitro-o-phenylenediamine, 6-nitro-1,2,3,4-tetrahydroquinone, 2-hydroxy-1,4-naphthoquinone, picramine acid and its salts, 2-amino-6-chloro-4-nitrophenol, 4-ethylamino-3-nitrobenzoic acid and 2-chloro-6-ethylamino-4-nitrophenol.

Suitable anionic direct drawing dyes may be selected from the group including Acid Yellow 1, Yellow 10, Acid Yellow 23, Acid Yellow 36, Acid Orange 7, Acid Red 33, Acid Red 52, Pigment Red 57:1, Acid Blue 7, Acid Green 50, Acid Violet 43, Acid Black 1, Acid Black 52, Bromophenol Blue and Tetrabromophenol Blue.

Suitable cationic direct drawing dyes are cationic triphenylmethane dyes, such as Basic Blue 7, Basic Blue 26, Basic Violet 2 and Basic Violet 14, aromatic systems substituted with a quaternary nitrogen group, such as Basic Yellow 57, Basic Red 76, Basic Blue 99, Basic Brown 16 and Basic Brown 17, cationic anthra-quinone dyes, such as HC Blue 16 (Bluequat B), and direct drawing dyes containing a heterocycle which has at least one quaternary nitrogen atom, in particular Basic Yellow 87, Basic Orange 31 and Basic Red 51. The cationic direct drawing dyes marketed under the trademark Arianor are also suitable cationic direct drawing dyes as contemplated herein.

Dyeing processes on keratin fibers usually take place in an alkaline environment. In order to protect the keratin fibers and also the skin as far as possible, however, the adjustment of a too high pH value is not desirable. Therefore, it is preferable if the pH value of agent (B) is between about 7 and about 11, especially between about 8 and about 10.5. For the purposes of the present disclosure, the pH values are pH values measured at a temperature of 22° C.

Preparation (B) may contain at least one alkalizing agent. The alkalizing agents usable for adjusting the preferred pH value as contemplated herein may be selected from the group formed from ammonia, alkanolamines, basic amino acids, as well as inorganic alkalizing agents such as (earth) alkali metal hydroxides, (earth) alkali metal metasilicates, (earth) alkali metal phosphates and (earth) alkali metal hydrogen phosphates. Preferred inorganic alkalizing agents are magnesium carbonate, sodium hydroxide, potassium hydroxide, sodium silicate and sodium metasilicate. Organic alkalizing agents which may be used as contemplated herein are preferably selected from monoethanolamine, 2-amino-2-methyl-propanol and triethanolamine The basic amino acids usable as alkalizing agents as contemplated herein are preferably selected from the group formed by arginine, lysine, ornithine and histidine, especially preferably arginine. However, in the course of the investigation into the present disclosure, it was found that further agents preferred as contemplated herein are exemplified by the fact that they additionally contain an organic alkalizing agent. An embodiment of the first subject-matter of the present disclosure is exemplified in that the agent additionally contains at least one alkalizing agent which is selected from the group formed by ammonia, alkanolamines and basic amino acids, in particular ammonia, monoethanolamine and arginine or its compatible salts.

Preparation (B) may also contain additional active ingredients, auxiliary substances and additives, e.g. one or more fatty components from the group of C₁₂-C₃₀ fatty alcohols, C₁₂-C₃₀ fatty acid triglycerides, C₁₂-C₃₀ fatty acid monoglycerides, C₁₂-C₃₀ fatty acid diglycerides and/or hydrocarbons.

Preferably, a surface-active substance may be added to preparation (B), such surface-active substances being designated as surfactants or emulsifiers depending on the area of application: they are preferably selected from anionic, zwitterionic, amphoteric and non-ionic surfactants and emulsifiers.

Preparation (B) preferably contains at least one anionic surfactant. Preferred anionic surfactants are fatty acids, alkyl sulfates, alkyl ether sulfates and ether carboxylic acids with from about 10 to about 20 C atoms in the alkyl group and up to about 16 glycol ether groups in the molecule.

Furthermore, preparation (B) may additionally contain at least one zwitterionic surfactant. Preferred zwitterionic surfactants are betaines, N-alkyl-N,N-dimethylammonium-glycinate, N-acyl-aminopropyl-N,N-dimethylammoniumglycinate, and 2-alkyl-3-carboxymethyl-3-hydroxyethyl-imidazolines. A preferred zwitterionic surfactant is known under the INCI designation cocamidopropyl betaine.

Furthermore, it may be intended that preparation (B) contains at least one amphoteric surfactant. Preferred amphoteric surfactants are N-alkylglycines, N-alkylpropionic acids, N-alkylamino-butyric acids, N-alkyliminodipropionic acids, N-hydroxyethyl-N-alkylamidopropylglycines, N-alkyltaurines, N-alkylsarcosines, 2-alkylamino-propionic acids and alkylamino acetic acids. Particularly preferred amphoteric surfactants are N-coconut alkylaminopropionate, as coconut acylaminoethylamino-propionate and C₁₂-C₁₈-acyl sarcosine.

Furthermore, it has proved to be advantageous if preparation (B) contains further non-ionic surface-active substances. Preferred non-ionic surfactants are alkyl polyglycoside and alkylene oxide plant products on fatty alcohols and fatty acids with from about 2 to about 30 mol of ethylene oxide per mol of fatty alcohol or fatty acid. Preparations with excellent properties are also obtained if they contain fatty acid esters of ethoxylated glycerol as non-ionic surfactants.

The non-ionic, zwitterionic or amphoteric surfactants are used in proportions of from about 0.1 to about 45% by weight, preferably from about 1 to about 30% by weight and particularly preferably from about 1 to about 15% by weight, based on the total weight of preparation (B).

Preparation (B) may also contain at least one thickener. There are no restrictions in principle with regard to these thickening agents. Both organic and purely inorganic thickeners may be used. Suitable thickeners are anionic, synthetic polymers, cationic, synthetic polymers, naturally occurring thickeners, such as non-ionic guar gums, scleroglucan gums or xanthan gums, gum arabicum, ghatti gum, karaya gum, tragacanth gum, carrageenan gum, agar-agar, carob bean gum, pectins, alginates, starch fractions and derivatives such as amylose, amylo-pectin and dextrine, as well as cellulose derivatives such as methyl cellulose, carboxyalkyl celluloses and hydroxyalkyl celluloses, non-ionic, fully synthetic polymers such as polyvinyl alcohol or polyvinyl pyrrolidinone, as well as inorganic thickeners, in particular layer silicates such as bentonite, especially smectites such as montmorillonite or hectorite.

In addition, preparation (B) may contain other active ingredients, auxiliary substances and additives, such as non-ionic polymers such as vinylpyrrolidine/vinyl acrylat copolymers, polyvinylpyrrolidinone, vinylpyrrolidine/vinyl acetate copolymers, polyethylene glycols and poly-siloxanes, additional silicones such as volatile or non-volatile, straight-chain, branched or cyclic, crosslinked or non-crosslinked polyalkylsiloxanes (such as dimethicones or cyclomethicones), polyarylsiloxanes and/or polyalkylaryl siloxanes, in particular polysiloxanes with organofunctional groups, such as substituted or unsubstituted amines (amodimethicones), carboxyl, alkoxy and/or hydroxyl groups (dimethicone copolyols), linear polysiloxane(A)-polyoxyalkylene(B) block copolymers, grafted silicone polymers; cationic polymers such as quaternized cellulose ethers, polysiloxanes with quaternary groups, dimethyldiallylammonium chloride polymers, acrylamidedimethyldiallylammonium chloride copolymers, dimethylaminoethyl methacrylate-vinylate vinyl pyrrolidinone copolymers quaternized with diethyl sulfate, vinyl pyrrolidinone-imidazolium methochloride copolymers and quaternized polyvinyl alcohol, zwitterionic and amphoteric polymers, anionic polymers such as, for example, polyacrylic acids or cross-linked polyacrylic acids, structurants such as glucose, maleic acid and lactic acid, hair-conditioning compounds such as phospholipids, for example lecithin and cephalins, perfume oils, dimethyl iso-sorbide and cyclodextrins, fiber structure-improving active substances, in particular mono-, di- and oligo-saccharides such as glucose, galactose, fructose, frucit sugar and lactose, dyes for coloring the product, anti-dandruff active substances such as piroctone olamines, zinc omadine and climbazole, amino acids and oligopeptides, animal and/or vegetable-based protein hydrolysates, as well as in the form of their fatty acid condensation products or optionally anionically or cationically modified derivatives, fatty substances and vegetable oils, sunscreens and UV blockers, active ingredients such as panthenol, pantothenic acid, pantolactone, allantoin, pyrrolidinone carboxylic acids and their salts, and bisabolol, polyphenols, in particular hydroxy cinnamic acids, 6,7-dihydroxy coumarins, hydroxy benzoic acids, catechins, tannins, leucoanthocyanidins, anthocyanidins, flavanones, flavones and flavonols, ceramides or pseudo-ceramides, vitamins, provitamins and vitamin precursors, plant extracts, fats and waxes such as fatty alcohols, beeswax, montan wax and paraffins, swelling and penetrating agents such as glycerine, propylene glycol monoethyl ether, carbonates, hydrogen carbonates, guanidines, urea and primary, secondary and tertiary phosphates, anti-caking agents such as latex, styrene/PVP and styrene/acrylamide copolymers, pearlescent agents such as ethylene glycol mono- and distearate and PEG-3 distearate and pigments.

The selection of these additional substances will be made by the expert according to the desired properties of preparation (B) as well as the product as contemplated herein. With regard to further optional components and the quantities of these components used, explicit reference is made to the relevant handbooks known to the expert. The additional active ingredients and auxiliary substances are preferably used in preparation (B) in quantities of from about 0.0001 to about 25% by weight each, in particular from about 0.0005 to about 15% by weight, each based on the total weight of preparation (B).

The following examples explain the present disclosure without, however, restricting it:

### EXAMPLES

A 100 μm thick layer of silicon dioxide SiOₓ was evaporated onto a polyethylene terephthalate film layer with a thickness of 40 μm (micrometers). The SiOₓ layer was then painted over with approx. 3 g/m² of ORMOCER polymer and cured. A 70 μm (micrometer) thick layer of polyethylene was then applied to the ORMOCER layer. A packaging (VP) was produced from the film.

The following cosmetic compositions (KM) were used (all figures in percentage by weight).

| Ingredients | KM |
|---|---|
| Potassium hydroxide (50%) | 0.19 |
| Sodium benzoate | 0.04 |
| Dipicoline acid | 0.10 |
| Disodium pyrophosphate | 0.10 |
| 1-hydroxyethane-1,1-diphosphonic acid (60%) | 0.25 |
| Propandiol-1,2 | 1.0 |
| Oxidizing agent ¹⁾ | 12 |
| C₈-C₃₀ alcohol ²⁾ | 3.40 |
| Cationic surfactant ³⁾ | 0.39 |
| Non-ionic surfactant ⁴⁾ | 1.0 |
| Liquid cosmetic oil ⁵⁾ | 0.3 |
| Water | ad 100 |

¹⁾ preferably hydrogen peroxide, calculated to 100% H₂O₂
²⁾ preferably a mixture of linear C₁₄-C₁₈ alcohols, especially cetearyl alcohol
³⁾ preferably an alkylammonium salt, especially steartrimonium chloride
⁴⁾ preferably an attachment product of 40 mol ethylene oxide to hydrogenated castor oil, especially PEG-40 hydrogenated castor oil, or ceteareth-20
^{2),} ^{3),} ⁴⁾ the ingredients may also be contained as a commercially available raw product (Emulgade ® F) in the exemplary cosmetic composition (4.5% by weight).
⁵⁾ preferably paraffin oil

| Ingredients | KM |
|---|---|
| Potassium hydroxide (50%) | 0.19 |
| Sodium benzoate | 0.04 |
| Dipicoline acid | 0.10 |
| Disodium pyrophosphate | 0.10 |
| 1-hydroxyethane-1,1-diphosphonic acid (60%) | 0.25 |
| Oxidizing agent ¹⁾ | 9.1 |
| C₈-C₃₀ alcohol ²⁾ | 3.40 |
| Cationic surfactant ³⁾ | 0.39 |
| Non-ionic surfactant ⁴⁾ | 1.0 |
| Liquid cosmetic oil ⁵⁾ | 0.3 |
| Propandiol-1,2 | 1.0 |
| Water | ad 100 |

¹⁾ preferably hydrogen peroxide, calculated to 100% H₂O₂
²⁾ preferably a mixture of linear C₁₄-C₁₈ alcohols, especially cetearyl alcohol
³⁾ preferably an alkylammonium salt, especially steartrimonium chloride
⁴⁾ preferably an attachment product of 40 mol ethylene oxide to hydrogenated castor oil, especially PEG-40 hydrogenated castor oil, or ceteareth-20
⁵⁾ preferably paraffin oil

The cosmetic composition KMin was filled into the packaging (VP) described above. Then the packages were stored for 24 weeks at 40° C. The packages were not inflated or delaminated.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. Cosmetic product for modifying the natural color of keratinous fibers, comprising
(i) at least one packaging (VP) comprising at least one multi-layer film (F), wherein the multi-layer film (F) comprises at least one first polymer layer (P1), at least one second polymer layer (P2) and at least one barrier layer (BS), and
(ii) at least one cosmetic composition (KM) packaged in the packaging (VP) and omprising:
a) at least one oxidizing compound,
b) at least one C8-C30 alcohol, and
c) at least one non-ionic surfactant,
wherein the first polymer layer (P1) is formed from polyethylene terephthalate or polyethylene naphthalate, the second polymer layer (P2) is formed from a polyolefin, and the barrier layer (BS) is formed from a polyester provided with an SiOₓ layer.

2. Cosmetic product according to claim 1, wherein the first polymer layer (P1) has a layer thickness of from about 4 μm to about 50 μm, the second polymer layer (P2) has a layer thickness of from about 20 μm to about 150 μm and/or the layer thickness of the barrier layer (BS) is from about 4 μm to about 20μm.

3. Cosmetic product according to claim 1, wherein the multi-layer film (F) has an oxygen transmission rate (OTR) at 23° C. and 50% relative humidity of from about 0.1 to about 5 cc/m²/d/bar, and a water vapor permeability at 38° C. and 100% relative humidity of from about 0.1 to about 5 g/m²d.

4. Cosmetic product according to claim 1, wherein the adhesive strength of the film is from about 0.1 to about 10 N/15 mm, and/or wherein the seal strength of the packaging (VP) is from about 10 to about 40 N/15 mm, under the conditions 150° C., 2.54 cm (1″) and 4 kg/cm².

5. Cosmetic product according to claim 1, wherein the at least one multi-layer film comprises the at least one barrier layer (BS) between the at least one first polymer layer (P1) and the at least one second polymer layer (P2).

6. Cosmetic product according to claim 1, wherein the first polymer layer (P1) forms the outer layer.

7. Cosmetic product according to claim 1, wherein the cosmetic composition (KM) has a pH value (measured at 20° C.) of between about pH 1.5 and about pH 5.0.

8. Cosmetic product according to claim 1, wherein the cosmetic composition (KM) comprises at least one oxidizing compound, in a total amount of from about 0.5 to about 20% by weight.

9. Cosmetic product according to claim 1, wherein the cosmetic composition (KM) comprises at least one non-ionic surfactant, in a total amount of from about 0.1 to about 5% by weight of the total amount of the cosmetic product (KM).

10. Cosmetic product according to claim 1, wherein the C₈-C₃₀ alcohol is a mixture of linear C₁₆-C₁₈ alcohols, which are included in the cosmetic composition (KM) in a total amount of from about 0.1 to about 10% by weight.

11. Cosmetic product according to claim 1, wherein the first polymer layer (P1) is formed from polyethylene terephthalate

12. Cosmetic product according to claim 1, wherein the second polymer layer (P2) is formed from polyethylene,

13. Cosmetic product according to claim 1, wherein the barrier layer (BS) is formed from a polyethylene terephthalate provided with an SiOₓ layer

14. Cosmetic product according to claim 1, wherein the first polymer layer (P1) has a layer thickness of from about 6 μm to about 20 μm.

15. Cosmetic product according to claim 14, wherein the second polymer layer (P2) has a layer thickness of from about 40 μm to 90 μm.

16. Cosmetic product according to claim 15, wherein the layer thickness of the barrier layer (BS) is from about 6 μm to about 15 μm.

17. Cosmetic product according to claim 1, wherein the cosmetic composition (KM) has a pH value (measured at 20° C.) of between about pH pH 2.5 and about pH 4.

18. Cosmetic product according to claim 1, wherein the cosmetic composition (KM) comprises a mixture of linear C₁₆-C₁₈ alcohols substituted with an average of 20 ethoxy groups, in a total amount of from about 0.12 to about 3.5% by weight, based on the total weight of the cosmetic composition (KM).

19. Cosmetic product according to claim 1, wherein the C₈-C₃₀ alcohol is a mixture of linear C₁₆-C₁₈ alcohols, which are included in the cosmetic composition (KM) in a total amount of from about 1.5 to about 5.0% by weight, based on the total weight of the cosmetic composition (KM).

20. Cosmetic product for modifying the natural color of keratinous fibers comprising
(i) at least one packaging (VP) comprising at least one multi-layer film (F), wherein the multi-layer film (F) comprises at least one first polymer layer (P1) formed from polyethylene terephthalate, at least one second polymer layer (P2) formed from polyethylene and at least one barrier layer (BS) formed from a polyethylene terephthalate provided with an SiOₓ layer, wherein the at least one multi-layer film comprises the at least one barrier layer (BS) between the at least one first polymer layer (P1) and the at least one second polymer layer (P2), and wherein the first polymer layer (P1) forms the outer layer, and
(ii) at least one cosmetic composition (KM) packaged in the packaging (VP) and comprising:
a) at least one oxidizing compound in a total amount of from about 1.5 to 15% by weight, relative to the total weight of the cosmetic composition (KM),
b) a mixture of linear C₁₆-C₁₈ alcohols in a total amount of from about 1.5 to about 5.0% by weight, based on the total weight of the cosmetic composition (KM), and
c) a mixture of linear C₁₆-C₁₈ alcohols substituted with an average of 20 ethoxy groups in a total amount of from about 0.50 to 1.5% by weight, based on the total weight of the cosmetic composition (KM).
